(19) United States
(12) Patent Application Publication
(10) Pub. No.: US 2006/0099266 A1
(43) Pub. Date: May 11, 2006

(54) Slow release formulation of clarithromycin

(76) Inventors: Rakhi H. Rohra, Maharashtra (IN); Mallika Tushakiran, Mumbai (IN); Chetan Doshi, Mumbai (IN); Virendra M. Khanapure, Thane (IN); Dinesh C. Nambiar, Mumbai (IN); Erich Zeisl, Wiesing (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/540,448

(22) PCT Filed: Dec. 22, 2003
(86) PCT No.: PCT/EP03/14755

§ 371 Date: Sep. 1, 2005

(30) Foreign Application Priority Data
Dec. 23, 2002 (GB) 02300034.1

(51) Int. Cl. A61K 31/7048; A61K 9/14

(52) U.S. Cl. 424/488; 514/28; 514/29

(57) ABSTRACT

The invention relates to an orally administrable pharmaceutical comprising an alginate matrix consisting of a water-soluble alginate salt and a complex salt of alginic acid, a macrolide, and an inorganic salt characterized in that the inorganic salt is capable of donating a proton and has a pKₐ value in water of 4.0 to 9.0.

The present invention is related to orally administrable pharmaceutical compositions of macrolide antibiotics. Macrolide antibiotics are known. The “Merck Index” (12^{th} ed., 1996) lists for instance erythromycin (no. 3720), roxithromycin (no. 8433), azithromycin (no. 946), josamycin (no. 5280), clarithromycin (no. 2400) and tylosin (no. 9963). Telithromycin is known from e.g. WO01/14393.

It is further known that drugs are advantageously orally administered in an alginate matrix comprising a water-soluble alginate and a complex salt of alginic acid as described in EP-A-188040 in order to achieve an extended release of the antibiotic so that the patients need to take the macrolide only once a day. This contributes a lot to the compliance of patients. However, one problem arising with the extended release formulations of macrolides in an alginate matrix is their poor solubility under alkaline conditions such as existing in the small intestine. Thus, resorption and bioavailability of orally administered macrolide antibiotics in extended release formulations are usually low in the small intestine. Therefore, it has been proposed in WO 97/22335 to include an organic acid such as citric acid into the extended release formulation of a poorly soluble basic drug in order to improve solubility and thus also bioavailability of the basic drug. However, these controlled release compositions do not purport to minimize the adverse effects related to gastrointestinal disorders including nausea and vomiting. In addition, said alginate matrix extended release formulations contain the organic acid such as citric acid in a molar ratio to the macrolide antibiotic of 1:1. As a consequence, considerable high amounts of organic acid is released from the formulation in the GI tract which is undesirable for patients with gastritis, ulcers and/or gastroesophagal reflux. As macrolides are often used in combination with proton pump-inhibitors such as for instance omeprazole, pantoprazole or lansoprazole, which are known to be very unstable under acidic conditions, it is not desirable to have released an organic acid from the alginate matrix formulation because it could contribute to the inactivation or degradation of the proton-pump inhibitor.

Thus, the aim of the present invention is to provide an improved orally administered alginate matrix formulation of a macrolide antibiotic which avoids a strong decrease of the pH value during release of the active ingredient.

This problem underlying the present invention is solved by adding to the extended release formulation comprising an alginate matrix an inorganic salt that is able to donate a proton and exhibits a pKₐ value in water from 4.0 to 9.0, preferably from 5.0 to 9.0, e.g. from 5.0 to 8.0.

Hence, in one aspect the present invention provides an orally administrable pharmaceutical composition comprising an alginate matrix consisting of a water-soluble alginate salt and a complex salt of alginic acid, a macrolide, and an inorganic salt characterized in that the inorganic salt is capable of donating a proton and has a pKₐ value in water of 4.0 to 9.0.

The alginate matrix is formed as described in EP-A-188040 and consists of a water-soluble alginate and a complex salt of alginic acid. The water soluble alginate in a composition according to the present invention is typically an alkali metal salt of alginic acid such as a potassium or a sodium salt, or a magnesium or an ammonium salt of alginic acid. Preferred is a sodium alginate.

A complex salt of alginic acid in a composition according to the present invention is typically a sodium-calcium complex salt of alginic acid wherein the amount of calcium is precisely controlled and which is self-gelling without the necessity of reacting with the stomach acid or additional calcium ions. While sodium-calcium alginate is the preferred complex salt of alginic acid used in the present invention, the sodium ions may be replaced by another cation that yields a water-soluble alginate such as those mentioned above, e.g. potassium, other alkali metal, magnesium or ammonium. The calcium ion may be replaced by another polybasic cation which yields an insoluble alginate salt, e.g. iron, strontium or barium. Magnesium is not suitable as a polybasic cation.

The weight ratio of soluble alginate to complex salt of alginic acid may vary from about 16:1 to 1:1, preferably from about 8:1 to 2:1. The same ratio applies to the ratio of sodium alginate to sodium-calcium-alginate. Typically, the amount of soluble alginate in a composition varies from about 6% to about 25% of the total weight of the composition, and the amount of the complex salt of alginic acid varies from about 0.5% to about 10% of the total weight of the composition.

A composition according to the present invention comprises a macrolide antibiotic. Suitable macrolide antibiotics are any basic macrolide antibiotic, for example basic macrolide antibiotics having a solubility in water of less than 33 g/l at room temperature. Suitable macrolides are in particular those mentioned above, i.e. erythromycin, roxithromycin, azithromycin, josamycin, clarithromycin, tylosin or telithromycin. In a preferred embodiment of the present invention the macrolide is clarithromycin. The amount of macrolide may vary in the composition from about 40%, preferably from about 50% to about 65%, preferably to about 75% of the total weight of the composition.

A composition according to the present invention comprises an inorganic salt that is capable of donating at least one proton and that has a pKₐ value in water at 25° C. of 4.0 to 9.0. The pKₐ value is defined as the negative logarithm of the dissociation constant Kₐ of an acidic compound dissolved in water at 25° C. and the pKₐ value is determined as conventional, e.g. according to known methods under standard conditions.

Preferably the pKₐ value of an inorganic salt in water according to the present invention is from 5.0 to 9.0, e.g. from 5.0 to 8.0. Suitable salts are for instance hydrogensulfates, e.g. of an alkali metal or an earthalkaline metal, dihydrogenphosphates, e.g. of an alkali metal or an earthalkaline metal, such as sodium dihydrogenphsophate, potassium dihydrogenphosphate, sodium hydrogensulfate or potassium hydrogensulfate. In a preferred embodiment a composition of the present invention comprises potassium dihydrogenphosphate as an inorganic salt which is capable to donate a proton and has a pKₐ value of 4.0 to 9.0. The inorganic salt is present in a composition according to the present invention in an amount corresponding to a ratio of inorganic salt: macrolide of 1:2 to 1:100 on a weight basis. Typically, a formulation according to the present application comprises about 20 to about 160 mg of the inorganic salt per 500 mg of the macrolide. Preferably, the inorganic salt is present in an amount of about 80 mg per 500 mg of the macrolide.

A composition according to the present invention may be any orally administrable pharmaceutical composition, in particular a tablet, a capsule or a pellet, such as a tablet. In a preferred embodiment, the composition is a once daily administrable formulation, e.g. a tablet for once daily administration regimen.

A composition according to the present invention, e.g. a tablet may comprise additional ingredients such as one or more further active drug compound(s) and/or pharmaceutically acceptable excipients such as conventionally used in the preparation of macrolide formulations, for example a binder, e.g. polyvinyl pyrrolidone, hydroxypropylcellulose, sodium carboxymethylcellulose, a filler, e.g. lactose, dicalciumphosphate, mannitol, starch, microcrystalline cellulose, a glidant/lubricant, e.g. talcum, magnesiumstearate, stearic acid, and/or a preservative, e.g. potassium sorbate. The composition may additionally comprise flavoring, coloring and light-protecting agents, e.g. vanillin, titanium dioxide. The amounts of excipients in the composition depend on the specific formulation and are as conventional in pharmaceutical formulations of macrolides. A tablet may be coated by conventional coating agents such as hydroxypropylmethylcellulose (e.g. Opadry®), e.g. in order to mask a bitter taste of the composition.

A composition according to the present invention may be prepared according to a known method, e.g. by a process comprising the steps of mixing a macrolide with a water-soluble alginate salt, a complex salt of alginic acid, an inorganic salt that has a pKₐ-value in aqueous solution of 4.0 to 9.0 and that is capable of donating a proton, and optionally with pharmaceutically acceptable excipients. The mixture may be granulated according to conventional granulation technology, e.g. by adding water, and by drying the obtained granules using conventional drying technology. The dried granules may optionally be resized, e.g. by sieving. In case the composition is a capsule, the granules are filled into a capsule, e.g. a gelatine capsule. In case the composition is a tablet, the granules may be mixed with glidants/lubricants and compressed into tablets analogous to, e.g. according to technologies as conventional. If desired, a tablet core may be coated with known coating agents analogously, e.g. according to known methods.

A unit dosage form of a composition of the present invention comprises from 100 mg to 2000 mg of a macrolide antibiotic. Preferably, a once daily administered dosage form comprises from 250 mg to 1000 mg of a macrolide antibiotic.

A composition according to the present invention avoids a strong pH decrease during release as it occurs with formulations according to the prior art comprising an organic acid, e.g. according to WO97/22335. Table 1 shows a comparison of the pH values during dissolution in water of a tablet comprising 500 mg clarithromycin and either
A) an equimolar amount of citric acid (as described in the prior art, e.g. WO97/22335), or

B) 80 mg of potassium dihydrogenphosphate (according to the present invention) under identical conditions:

TABLE 1
comparison of pH values in water during release of a tablet according to
the present invention (B) and a tablet according to prior art (A)

| | tablet A | |
|---|---|---|
| pH in water | comprising citric acid | tablet B comprising KH₂PO₄ |
| after 5 minutes | pH 4.9 | pH 6.1 |
| after 30 minutes | pH 4.5 | pH 6.3 |
| after 60 minutes | pH 4.4 | pH 6.4 |
| after 120 minutes | pH 4.3 | pH 6.5 |

This effect has special advantages when a macrolide antibiotic composition according to the present invention is combined with a proton pump inhibitor. Macrolide antibiotics are often combined with proton pump inhibitors, such as omeprazole, lansoprazole or pantoprazole in the treatment of gastritis, gastrointestinal ulcers and/or gastroesophageal reflux diseases in order to increase the pH of gastric juices. Proton pump inhibitors are tightly unstable under acidic conditions. Therefore, a composition according to the present invention may contribute to decrease the risk of inactivation or degradation of proton-pump inhibitors.

In addition, it is generally not desirable to introduce additional acidic compounds into gastric juices of patients suffering in gastritis, gastrointestinal ulcers and/or gastroesophageal reflux diseases. As can be seen from table 1, a composition of the present invention lowers the pH during release to a much less extent than macrolide antibiotic compositions according to the prior art which comprise an organic acid.

In a preferred embodiment, the present invention relates to a film coated tablet comprising 500 mg clarithromycin, from about 80 mg to about 150 mg sodium alginate, from about 5 mg to about 50 mg sodium-calcium alginate, from about 20 mg to about 160 mg potassium dihydrogen phosphate, from about 30 mg to about 60 mg povidone (K=30), from about 100 mg to about 300 mg lactose monohydrate, from about 100 mg to about 300 mg dicalcium phosphate, from about 20 mg to about 30 mg talcum, and from about 10 mg to about 20 mg magnesium stearate.

The following Example demonstrates the present invention but shall in no way be construed to limit its scope.

### EXAMPLE

A film coated tablet was prepared according to the following process:

| Sr. no. | Ingredient | mg/tab | g/batch |
|---|---|---|---|
| 1. | Clarithromycin | 500 | 5.000 |
| 2. | Sodium alginate (Manucol | 120 | 1.200 |
| | LKX) | | |
| 3. | Sodium-Calcium alginate | 30 | 300 |
| | (Kelset) | | |
| 4. | Potassium dihydrogen | 80 | 800 |
| | phosphate | | |
| 5. | Povidone (K = 30) | 30 | 300 |
| 6. | Lactose monohydrate | 120 | 1.200 |
| 8. | Talc | 30 | 300 |
| 9. | Magnesium stearate | 10 | 100 |
| 10. | Opadry yellow | 20 | 200 |
| | Film coated tablet wt. | 940 | |

Components 2, 3, 4, 5 and 6 were screened through a 425μ aperture screen. The sieved excipients were dry blended with the API (component 1) in a high shear mixer. The blended material was granulated using water to obtain granules. The granules were dried in a fluidised bed drier at 60° C. until the granules had a moisture content of less than 4.0% w/w. (105° C., 15 min). The dried granules were resized using a 850μ aperture screen and then blended with lubricants (component 8 and 9) in a double-cone blender. The lubricated tablet blend was compressed into tablets using a rotary tablet machine. The core tablets were coated in a conventional perforated coating pan using an aqueous suspension of Opadry.

The controlled release of clarithromycin was determined by the in vitro dissolution profile in comparison to a tablet of clarithromycin on the market:

Medium: 900 ml Acetate buffer pH 5.0, 50 RPM, Paddle type

| | tablet according to the | |
|---|---|---|
| time [h] | present invention | tablet according to WO97/22335 |
| 2 | 9 | 6 |
| 4 | 21 | 18 |
| 6 | 32 | 31 |
| 8 | 43 | 43 |
| 10 | 52 | 53 |
| 12 | 60 | 61 |
| 16 | 71 | 72 |
| 24 | 87 | 86 |

The decrease of pH value during dissolution in water of a tablet obtained from the above described process is compared under identical conditions to a dissolution of a tablet according to WO97/22335 comprising 500 mg clarithromycin and an equimolar amount of citric acid:

| | tablet | |
|---|---|---|
| pH in water | comprising citric acid | tablet comprising KH₂PO₄ |
| after 5 minutes | pH 4.9 | pH 6.1 |
| after 30 minutes | pH 4.5 | pH 6.3 |
| after 60 minutes | pH 4.4 | pH 6.4 |
| after 120 minutes | pH 4.3 | pH 6.5 |

1. An orally administrable pharmaceutical composition comprising an alginate matrix consisting of a water-soluble alginate salt and a complex salt of alginic acid, a macrolide, and an inorganic salt wherein the inorganic salt is capable of donating a proton and has a pKₐ value in water of 4.0 to 9.0.

2. A composition according to claim 1 wherein the water-soluble alginate salt is an alkali metal salt of an alginate.

3. A composition according to claim 1 wherein the complex salt of alginic acid is sodium-calcium alginate.

4. A composition according to claim 1 wherein the macrolide is selected from the group consisting of erythromycin, roxithromycin, azithromycin, josamycin, telithromycin, clarithromycin and tylosin.

5. A composition according to claim 4 wherein the macrolide is clarithromycin.

6. A composition according to claim 1 wherein the inorganic salt has a pKₐ value of 5.0 to 8.0.

7. A composition according to claim 1 wherein the inorganic salt is an alkali metal or earth-alkaline dihydrogenphosphate or hydrogensulfate.

8. A composition according to claim 1 wherein the ratio of inorganic salt: macrolide is from 1:2 to 1:100 on a weight basis.

9. A composition according to claim 1 wherein the pharmaceutical composition is a tablet for once daily administration.

10. Process for preparing a once daily orally administrable formulation of a macrolide by mixing the macrolide with a water-soluble alginate salt, a complex salt of alginic acid, and an inorganic salt, wherein the inorganic salt has a pKₐ value in water of 4.0 to 9.0 and is capable of donating a proton.
